(19) United States
(12) Patent Application Publication
(10) Pub. No.: US 2010/0020917 A1
(43) Pub. Date: Jan. 28, 2010

(54) X-RAY SYSTEM, AND METHOD FOR GENERATING X-RAY IMAGES

(75) Inventor: Horacio Sergio Gagliano, Buonas (CH)

Correspondence Address: THE NATH LAW GROUP, 112 South West Street, Alexandria, VA, 22314, US

(73) Assignee: SWISSRAY INTERNATIONAL INC., Elizabeth, NJ (US)

(21) Appl. No.: 12/312,645

(22) PCT Filed: Nov. 23, 2006
(86) PCT No.: PCT/EP2006/068807

§ 371 Date: Jul. 31, 2009

(51) Int. Cl. A61B 6/03; G01N 23/04

(52) U.S. Cl. 378/4

(57) ABSTRACT

An x-ray system for generating x-ray images comprises an x-ray transmitting/receiving device, and an input arrangement (5) for determining the exposure parameters of an x-ray process, wherein the object to be x-rayed can be depicted on a screen surface (10) of a visual display unit (6) as a virtual model (12) in at least one position. The display unit (6) is operationally connected with the input arrangement (5) such that the exposure parameters can be adjusted as a function of the position of the model (12). In order to consider a further position of the object to be x-rayed, the display unit (6) can be actuated by means of input means (7) of a data processing system (11) such that at least one further position of the model (12) can be adjusted on the screen surface (10).

The invention relates to an X-ray system for generating X-ray images in accordance with the preamble of claim 1, and a method for generating preferably digital X-ray images in accordance with the preamble of claim 10.

Methods and a device of digital radiographic technology have been known for a relatively long time and are conventional, reference being made here (in place of many) to WO 96/22654 and WO 97/20231 in an exemplary manner.

The prior art discloses that persons to be X-rayed can be displayed on a screen as virtual model, with a selection of body parts to be X-rayed being prescribed in the model. An X-ray source can be adjusted on the basis of a selected body part. It is also known that certain patient-specific parameters, in particular the weight of the person, have to be taken into account in the determination of the exposure parameters of the X-ray procedure. Such patient-specific data can be input by means of an input arrangement. However, the fact that the correct setting of the X-ray transmitter/receiver unit still depends strongly on the experience and judgment of the operating persons still holds true today. One problem in particular lies in the fact that only one position of the model assigned to the patient is displayed on the screen and it is used only for selecting the body parts to be X-rayed. Hence it is difficult to correctly and easily adjust the corresponding exposure parameters for this and other positions of the patient.

It is therefore an object of the invention to avoid the disadvantages of that which is known; in particular, it is an object of the invention to develop an X-ray system of the type mentioned initially, by means of which X-ray images of an object to be X-rayed can easily be generated in different positions. The X-ray system should be distinguished by easy operator guidance. Furthermore, it should make it possible to increase the number of X-ray images per unit time. Thereafter, error rates as a result of incorrect settings and the like should be reduced. According to the invention, these objects are achieved by an X-ray system having the features of claim 1.

The X-ray transmitter/receiver unit has a radiation source for emitting X-rays and an X-ray imaging device. The X-ray imaging device can preferably be a digital X-ray image sensor. However, it goes without saying that, alternatively, a conventionally designed X-ray image sensor is also conceivable.

A virtual model on the screen is associated with the physical object to be X-rayed. In the case of persons to be X-rayed, this model can be a genderless representation of a human. It goes without saying that it is conceivable to select appropriate models by means of input means, depending on the sex of the person to be X-rayed. However, it is also possible that animals are the objects to be X-rayed. However, it goes without saying that the invention is not limited to the field of application of human medicine or veterinary medicine.

In addition to other parameters, setting of the X-ray transmitter/receiver unit also depends on the position of the object to be X-rayed. Adjusting and displaying further positions of the preferably same model on the screen makes intuitive operation possible. The exposure parameters of a respective X-ray procedure, in particular the exposure parameters for controlling the radiation dose, can easily be specified in this manner. Since data relating to the position of the model can be retrievable from a position-data storage, certain settings can thereby be influenced directly on the generator without the operating person having to intervene manually.

The input means for adjusting the position of the model can be directly or indirectly connected to the display device. By way of example, one such input means can be a mouse, by means of which boxes or buttons, which can be activated using a cursor, can be selected on the screen. However, the input means can also be selection switches on an operator console. A keyboard is also feasible. The use of a touch-reactive screen (so-called “touch screens”) would also be conceivable. An image processing algorithm of the data processing system can be activated with the aid of the input means, which algorithm generates the image with the model, which shows the position of the model, on the screen.

In a first embodiment, the data processing system can comprise means for the animated display of the model during a transition from one position to a next position. The advantage of animating the model is that a positional change can advantageously be displayed visually. Using the animation, the operating staff of the X-ray system could also pass simple instructions to the person to be X-rayed.

The display device can be designed to display the model in different predetermined positions on a screen. On the basis of these positions of the model, the object to be X-rayed can be accordingly aligned with reference to the X-ray transmitter/receiver unit. In the process, a person to be X-rayed in general only has to follow the instructions of the operating staff.

It is particularly advantageous if the model can be displayed in a front view, a back view and at least one side view, it being possible to select the respective position by means of input means. Such an arrangement is in particular suited to imaging standing persons, with the model in this case accordingly also being displayed in a standing fashion on the screen. By in each case respectively using at least three model positions, but preferably four model positions (front view, back view, two side views), in a certain state (e.g. standing state) in each case, the risk of erroneous settings can be avoided. However, the model could additionally or alternatively also be displayed on the screen in different states. By way of example, a lying state would be a different state, in which the person to be X-rayed would place himself or would have to be placed on the stomach or on the back on an X-ray table. In this case, the model would have to preferably be displayed on the screen with a horizontal alignment instead of a vertical alignment.

The display device can display a model which has selection regions respectively assigned to a body part or an organ. The selection region to be X-rayed can be selectable by means of input means, the input arrangement being designed to determine the exposure parameters of the X-ray procedure as a function of the selected selection region. It is possible to use the same input means or different input means, which are assigned to the positioning of the model, as input means for selecting the selection region. As a result of selection regions being provided in preferably every position of the model, it is possible to optimize the exposure parameters for the X-ray procedure for the respective position in a particularly simple manner. Furthermore, the operation guidance is significantly simplified and possible sources of error are reduced in this manner.

In a further embodiment, the input arrangement can be coupled to a patient-specific interface so as to take into account patient-specific data for the determination of the exposure parameters of the X-ray procedure. By way of example, this interface can connect the input arrangement to a database for storing this data, which database contains all relevant patient-specific data. In addition or as an alternative to the patient database, further patient-specific data can be input into the input arrangement by means of input means such as a keyboard. Here, the input arrangement is preferably designed such that newly input data can be stored in the abovementioned patient-specific database. The superposition of the X-ray transmitter/receiver unit control, in particular the control of the radiation source, with patient-specific data leads to a significant optimization of the X-ray procedure. This is because the radiation dose also substantially depends on the age and the constitution of the patient. Furthermore, alternatively, or preferably additionally, the body mass index (BMI) of the patient should be taken into account. The BMI can be calculated from the weight and height of the patient. By way of example, to this end, the X-ray system could be equipped with corresponding mass sensors which are connected to the input arrangement.

If the X-ray transmitter/receiver unit is adjusted correctly and the patient is positioned correctly, the digital X-ray image can be generated. The data thereof in turn can be stored with respect to body part, radiation intensity etc., and a patient-specific patient-data storage can be complemented by the corresponding data. In the process, the patient-specific data is updated in some fashion, so that the optimal data is always available for the next X-ray procedure with the same patient. Overall, as a result of this, the erroneous irradiation rate and hence the average irradiation dose per patient can also be reduced.

The input arrangement can be connected or connectable to a database via an interface so as to take into account risk factors for the determination of the exposure parameters of the X-ray procedure. By using such risk factors in addition or if need be only alternatively to the patient-specific data, it is possible to further optimize an X-ray procedure with respect to the object to be X-rayed. By way of example, the database can be an osteoporosis-statistics database, from which osteoporosis risk factors are retrievable. Here, osteoporosis risk factors can substantially depend on the age, sex and ethnicity of the person to be X-rayed. Such an osteoporosis risk factor can be used by an appropriate algorithm for determining the exposure parameters.

In order to generate X-ray images of a person to be X-rayed, it can be advantageous if the database is an osteoporosis-statistics database for determining osteoporosis risk factors, the input arrangement being designed such that the osteoporosis risk factors can be selected from the osteoporosis-statistics database on the basis of the age and the sex of the person.

The input arrangement can be designed such that a selection dialog box for pathology-specific parameters can be displayed on the screen. In the process, it is possible to select at least one pathology-specific parameter by means of input means so as to take this parameter into account for the determination of the exposure parameters of the X-ray procedure. The use of pathology-specific parameters can further optimize the X-ray procedure. X-ray images generated as a function of pathology-specific parameters are distinguished by a significantly improved image quality for subsequent diagnoses. By way of example, the selection dialog box for pathology-specific parameters can have selectable boxes or buttons, with each box or each button in each case being assigned to a specific type of pathology (e.g. pneumonia, tuberculosis, cardio, osteoporosis, tumor, orthopedics, etc.). Furthermore, pathology-specific parameters can take into account the presence of casts, implants or dressing materials, it being possible for the exposure parameters also to depend on these. There is a correlation between the pathology-specific data and the radiation-specific data for actuating the X-ray transmitter/receiver unit.

With respect to the method, the objects set in accordance with the invention are achieved by a method which has the features of claim 10. The method is characterized in that the respective position of the object to be X-rayed is adjusted according to the model of the screen assigned to the object in order to be displayed on the display device by means of input means. In contrast to the known prior art, in which a model is only displayed in one position, different positions of preferably the same model can be set, as a result of which the position of the object to be X-rayed can be taken into account in a simple manner for specifying the exposure parameters of the X-ray procedure.

It is advantageous if a number of X-ray images are made in sequence in different positions. Thanks to the model displayed on the screen in different positions, the operating staff does not have to individually apply new settings manually to the X-ray system each time. The efficiency of the radiographic method can be significantly increased in this manner.

It can be advantageous in an embodiment if a positional change of the model is animated. When a control signal is received as a result of an input means being activated, the positional change can easily be animated by an animation algorithm and can be displayed on the screen.

It can be particularly advantageous if, in order to record X-ray images of a standing person, the model is turned about its longitudinal axis on the screen during the transition from one position to a next position. In this case, a model in a three-dimensional representation is preferably used. This transition can in each case take place in individual steps, in which the rotation is effected in each case through 90° (90° steps). Furthermore, the model could be animated in such a way that, during the rotation, it at least carries out the movements of the legs in an approximately natural manner.

With respect to the method, it can furthermore be advantageous if a pathology-specific parameter, which is taken into account when specifying the exposure parameters of an X-ray procedure, is selected by means of input means on the basis of a selection of pathology-specific parameters. Hence, the method enables better evaluation of the generated X-ray images.

Further individual features and advantages of the invention result from the following description of exemplary embodiments and from the drawings, in which

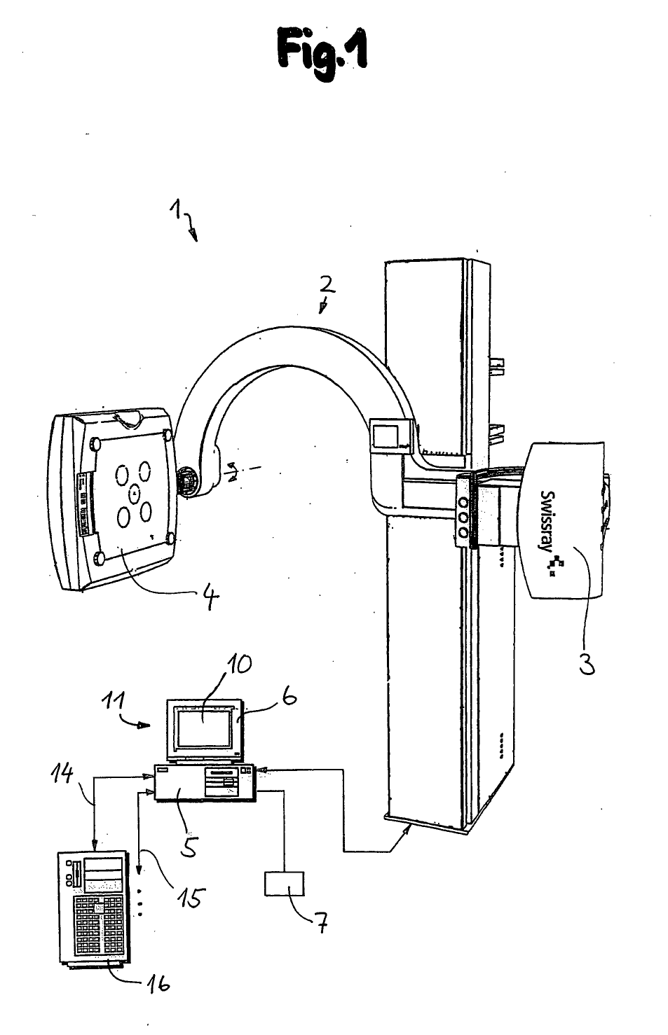

FIG. 1 shows an illustration of an X-ray system according to the invention with a perspective illustration of an X-ray transmitter/receiver unit and a schematic illustration of an input arrangement,

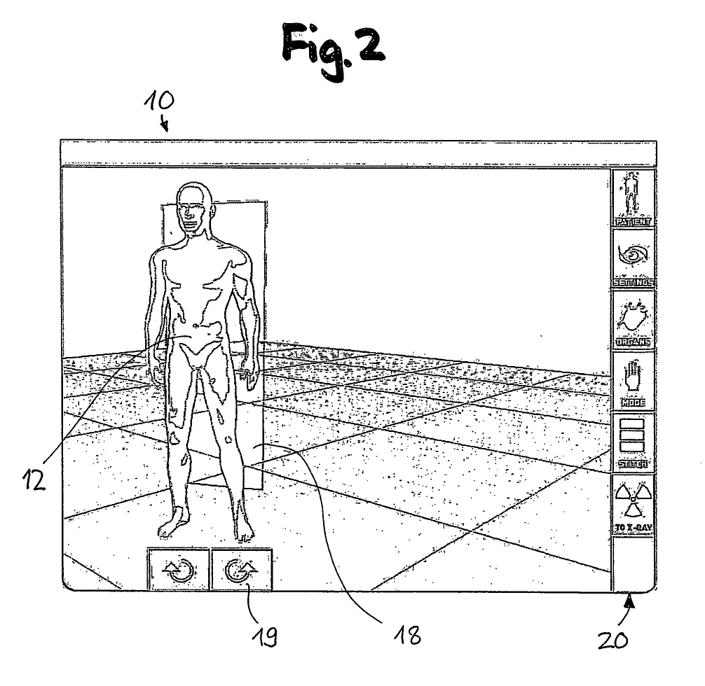

FIG. 2 shows a screen with a model in a front view in accordance with a first exemplary embodiment,

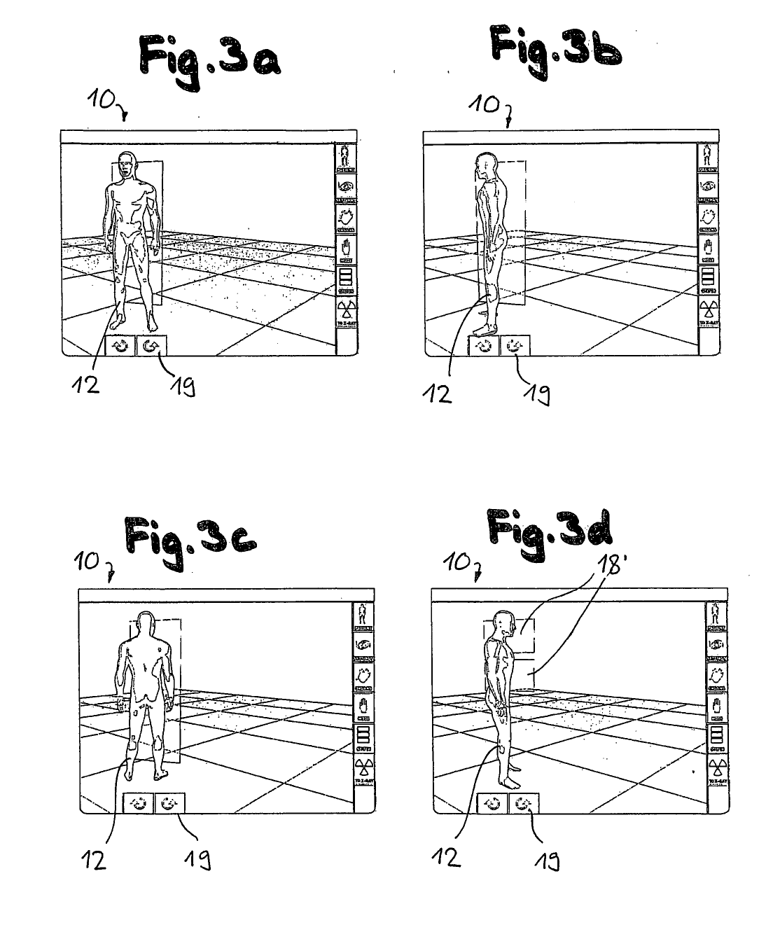

FIG. 3a shows a reduced illustration of the screen in accordance with FIG. 2,

FIGS. 3b/3c/3d show the screen with the model in accordance with FIG. 3a, but in different positions (side view/back view/side view),

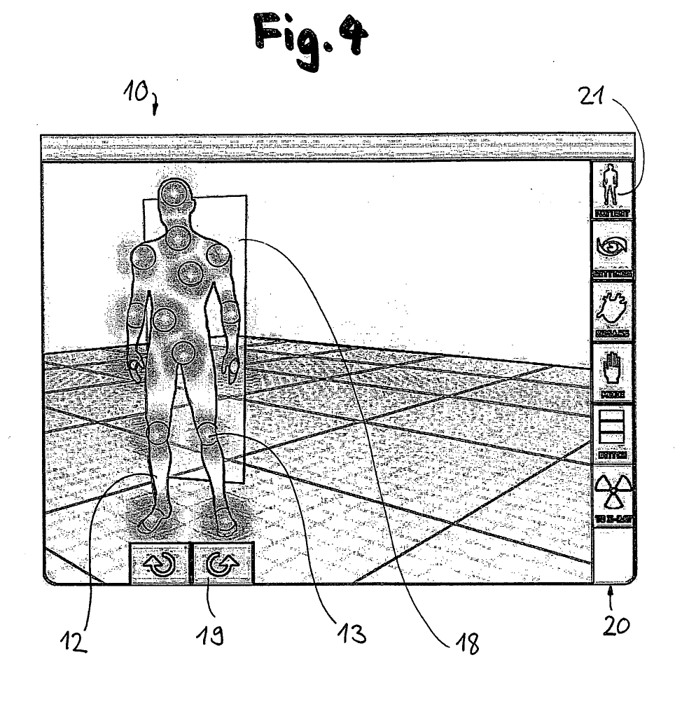

FIG. 4 shows the screen in accordance with FIG. 2 with a model with selection regions,

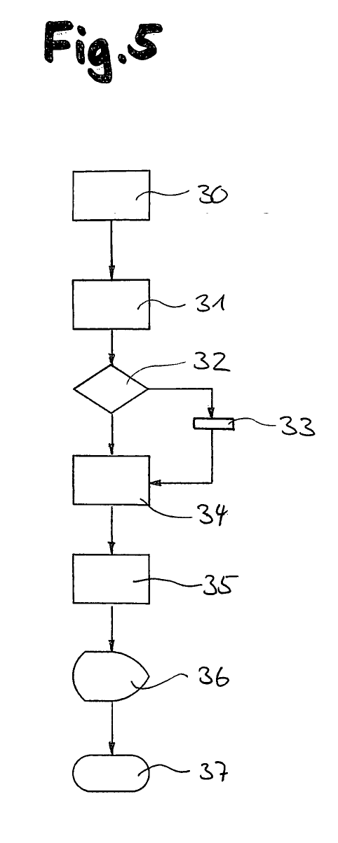

FIG. 5 shows a flowchart for a method sequence in an X-ray procedure,

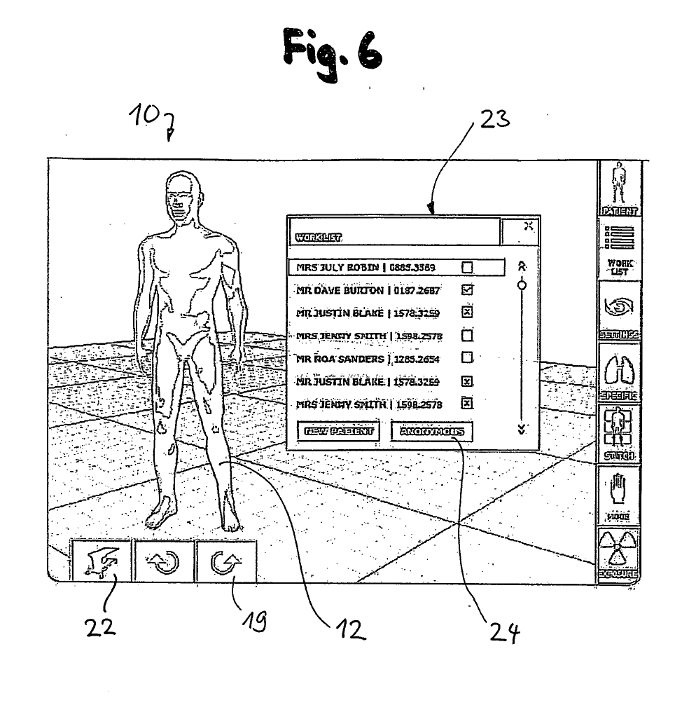

FIG. 6 shows a screen in accordance with a second exemplary embodiment with a patient selection box,

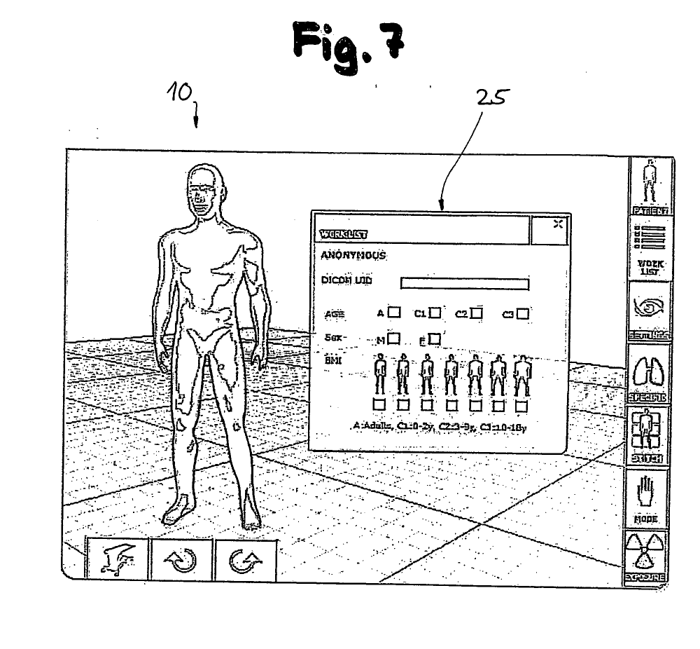

FIG. 7 shows the screen in accordance with FIG. 6, but with a patient-specific dialog box for still unknown persons,

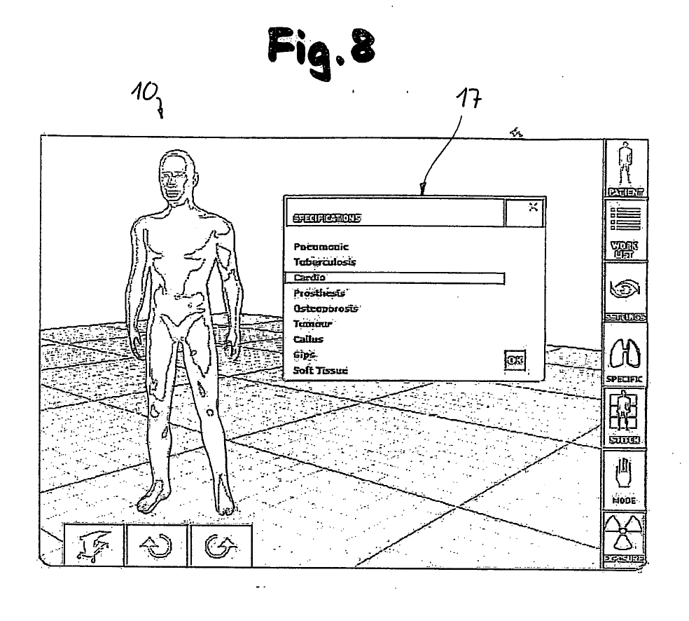

FIG. 8 shows the screen with a selection dialog box for pathology-specific parameters, and

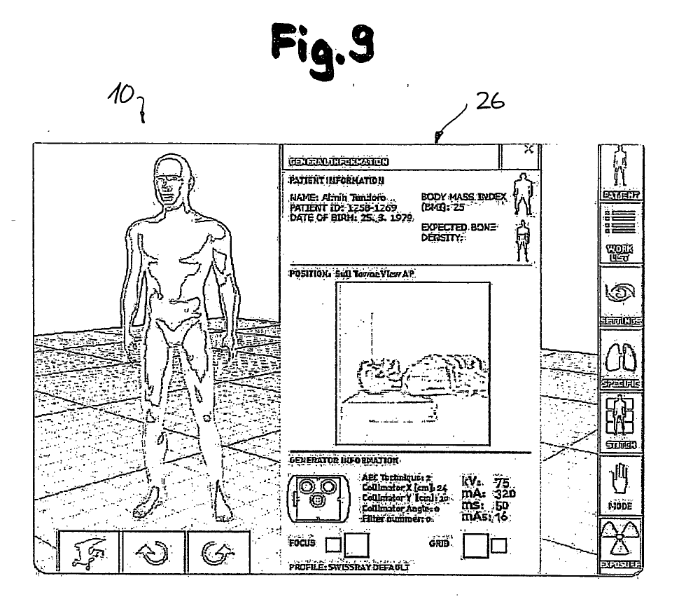

FIG. 9 shows the screen with a display box.

As illustrated in FIG. 1, an X-ray system designated by 1 comprises an X-ray transmitter/receiver unit 2 by means of which X-ray images can be generated using digital radiographic technology. To this end, it has a radiation source 3 for emitting X-rays and a digital X-ray image sensor. A comparable X-ray unit is disclosed, for example, in WO 2006/111202. In digital radiography, the image can be digitized directly, as a result of which the X-ray procedure as such is significantly accelerated. In accordance with another digital technique, an image is stored meta-stably on a phosphor plate and read by means of a laser. However, in principle, an X-ray unit from conventional radiography could also be suitable for the X-ray system according to invention described below in that case, the digital X-ray image sensor would have to be replaced by a conventional X-ray imaging device. In such X-ray imaging devices, the X-ray image would be recorded on X-ray film. With the exception of specifying the exposure parameters for an X-ray procedure according to the invention, the X-ray procedure proceeds in a known fashion.

The X-ray unit 2 is connected to an input arrangement 5 by means of which the exposure parameters of an X-ray procedure can be specified. In particular, the radiation dose, and preferably the irradiation duration as well, can be determined for an X-ray procedure with the aid of the input arrangement 5. FIG. 1 shows that a data processing system 11 with the input arrangement 5 furthermore comprises a visual display device 6. This display device 6 can be a monitor, on which a screen 10 can be observed. Data can be input and/or selected by means of input means indicated by 7. The input arrangement 5 is furthermore connected to a database of, for example, a database server via an interface. 15 indicates a further interface in order to take into account certain data for determining the exposure parameters of the X-ray procedure. Such data could, for example, also be retrieved via the Internet or an intranet. The data could, for example, be input using a mouse, a keyboard, selection switches, touch-reactive screens (touch screens) or other input means. Exposure parameters of an X-ray procedure can be specified using the input arrangement 5 of the X-ray system 1, with it being possible for an object to be X-rayed to be imaged on the screen 10 as a virtual model in at least one position. The display device 6 is operatively connected to the input arrangement 5, as a result of which the exposure parameters can be adjusted depending on the position of the model.

FIG. 2 illustrates a screen 10 of a display device. A three-dimensional model 12 of person to be X-rayed is displayed on the screen in a front view. This front view shows-the relative position of the person to be X-rayed to the X-ray unit, this position being attained in a simplified manner by referring to the imaging or sensor device which is displayed on the screen 10 as a rectangular imaging surface 18 in an indicative manner. FIG. 2 shows that the person to be X-rayed is implemented as a genderless model of a human. However, it goes without saying that it would also be conceivable—depending on the purpose of the application—to use different models (e.g. woman or man, animals, etc.). By selecting buttons 19, the model 12 can be rotated clockwise or anticlockwise, as desired, with respect to the imaging surface 18 (cf. following FIGS. 3a to 3d). Hence, the screen 10 directly or indirectly becomes an input unit and the source of information for the correct positioning of the patient.

A menu bar with buttons can be seen on the right edge of the screen 10, which buttons are labeled with “PATIENT”, “SETTINGS”, “ORGANS”, “MODE”, “STITCH”, and “X-RAY”. By way of example, in the “SETTINGS” menu, the treating medical practitioner or other operating staff can define certain requirements regarding the image quality, such as the image contrast, etc., which can be stored in an appropriate data storage.

The model 12 is illustrated in different positions in FIGS. 3a to 3d. The different positions can be adjusted in respectively 90° steps by rotating the model by executing a control command by means of an input means, for example by direct selection of the control field 19. As soon as a desired position of the model 12 is attained, the person to be X-rayed is instructed to also take up this position. However, it goes without saying that it would also be conceivable to position the person appropriately by means of technical implements or, for example, with the aid of hospital staff. Once the person is correctly positioned in relation to the X-ray transmitter/receiver unit, the X-ray procedure can be continued.

When a control signal is received as a result of activating the control field 19, a positional change is animated by means of an animation algorithm and displayed on the screen 10. The data processing system can be actuated by the control field or other input means such that further positions of the model can be set on the screen. This position data is processed by the data processing system and the exposure parameters are adjusted as a function of the respective position of the model.

The side view in accordance with FIG. 3d shows that, contrary to the previously shown screens, the imaging surface is composed of two imaging surface sections 18′. The X-ray images respectively assigned to these sections 18′ could be recombined by means of a stitching method.

FIG. 4 basically shows the same screen as FIG. 2, in which selectable selection regions 13 are highlighted by means of circles. The selection regions can, for example, also be highlighted visually by specific colors and/or brightness values. The selection regions are assigned to a body part or an organ of a person to be X-rayed. By selecting a selection region (e.g. the knee) by means of input means, the exposure parameters can be determined taking into account the selected body part or organ. The selection regions could, for example, be selected with the aid of a mouse and input into the input arrangement. However, it goes without saying that other input means would also be conceivable here. Although only FIG. 4 shows a model 12 with selection regions 13, it is particularly advantageous if the other positional displays of the model (cf. FIGS. 3b/3c/3d) were also to have such selection regions. Overall, this data selection also influences the X-ray transmitter/receiver unit control, the position-specific data additionally being superposed.

It is conceivable that the selection of a selection region 13 could in certain cases only be a pre-selection of a body region. After the pre-selection, a refined-selection box (not shown) with further selection options could appear on the screen.

FIG. 5 illustrates a flowchart for illustrating a possible method sequence for an X-ray procedure for generating an X-ray image. A desired position of the model for the person to be X-rayed is selected in a first step 30. Once the person has been brought into this position, a region to be X-rayed is selected in a second step 31 on the basis of the selection regions in the model (cf. FIG. 4). Thereafter, patient-specific data can be input in a next step. Depending on whether or not a patient is already included in a patient acquisition system (32), patient-specific data must first of all be acquired by an input step 33. Otherwise the patient can be selected from a list of patients. Once the relevant patient-specific data has been determined in method step 34, pathology-specific data, on which the exposure parameters may also depend, can be selected in a next step 35. Then the most important data can be displayed in an overview on a display box (step 36). Now the patient can be irradiated in step 37. The process is recommenced (back to step 30) for a subsequent image of the patient, in particular for sequential imaging of the patient. It goes without saying that the order as detailed above does not necessarily have to be adhered to. Then it could be possible for the execution of certain steps to be dispensed with if necessary. However, carrying out step 30 (selection of a desired position of the model) is mandatory in the case of the method according to the invention. Step 31 (selection of a selection region on the model) should preferably also be part of the method. The remaining steps 32 to 36 could be only optional parts of the method.

FIG. 6 shows a screen 10 that an observer could come across whilst executing step 32 in accordance with FIG. 5. A patient selection box 23 is displayed on the screen as a new window. It can be seen that the selection box 23 comprises a list of persons who can be selected in case of agreement. From the patient selection box 23, patient names from a patient storage can be retrieved and selected.

In the case where there is no agreement, the patient data must be newly acquired. This can be effected in two ways: selecting the “NEW PATIENT” button would open an input box (not shown), in which the individual data of a known person would have to be input into a corresponding template. In the case of anonymous patients (e.g. unconscious casualties without identification), the button “ANONYMOUS” would have to be selected, after which a selection box 25 in accordance with FIG. 7 would appear on the screen 10. Here, prescribed selection boxes are provided, by means of which the age, sex and BMI (body mass index) could be acquired. It can be seen that smaller two-dimensional figures, which are each assigned to a particular BMI value, are displayed in window 25 as an aid for determining the BMI. This makes it possible for the operating staff to estimate the BMI in a simple manner. The data retrieved in this manner defines certain settings of the X-ray unit for the determination of the exposure parameters.

On the right edge of the screen 10, in accordance with the exemplary embodiment of FIG. 6, a menu bar with “PATIENT”, “SETTINGS”, “WORK LIST”, “SETTINGS”, “SPECIFIC”, “STITCH”, “MODE”, and “X-RAY” is visible. Briefly, the individual menus can have the following functions: PATIENT: data check and possible input; WORK LIST: list of patients, for example from a network (hospital network or the like). Here, it is also possible to, as described above, add patients (known or anonymous); SETTINGS: check the generator settings; SPECIFIC: differential radiography (pathological images); STITCH: activate stitching; MODE: toggle between manual/auto mode; X-RAY: exposure stand-by for irradiating the person to be X-rayed.

A pathology-specific selection box 17 can be seen on the screen 10 in FIG. 8. By way of example, the “CARDIO” selection can be selected from a predetermined list for cardio-images.

In FIG. 9, a further window 26 can be seen on the screen 10. The window 26 contains patient-specific information (“PATIENT INFORMATION”), information regarding the position and information for setting the generator of the radiation source.

1-16. (canceled)

17. An X-ray system for generating X-ray images, having an X-ray transmitter/receiver unit comprising a radiation source (3) for emitting X-rays and a preferably digital X-ray image sensor (4) and comprising an input arrangement (5) for specifying the exposure parameters of an X-ray procedure, in particular for controlling the radiation dose, it being possible to image the object to be X-rayed on a screen (10) of a visual display device (6) as a virtual model (12) in at least one position, the display device (6) being operatively connected to the input arrangement (5) such that the exposure parameters can be adjusted depending on the position of the model (12), characterized
in that,
in order to take into account a further position of the object to be X-rayed by means of input means (7) of a data processing system (11), the display device (6) can be actuated such that at least one further position of the model (12) can be displayed on the screen (10).

18. The X-ray system as claimed in claim 17, characterized in that the data processing system (11) comprises means for the animated display of the model (12) during a transition from one position to a next position.

19. The X-ray system as claimed in claim 17, characterized in that the display device (6) is designed to display the model (12) in different predetermined positions on a screen (10).

20. The X-ray system as claimed in claim 19, characterized in that the model (12) can be displayed on the screen (10) in a front view, a back view and at least one side view in a two-dimensional or three-dimensional representation, it being possible to select the respective view by means of input means (7).

21. The X-ray system as claimed in claim 17, characterized in that the display device (6) can display a model (12) which has selection regions (13) assigned to a human or animal body part or organ, and in that the selection region to be X-rayed can be selected by means of input means (7), the input arrangement (5) being designed to determine the exposure parameters of the X-ray procedure as a function of the selection region.

22. The X-ray system as claimed in claim 17, characterized in that the input arrangement (5) is coupled to a patient-specific interface (14) so as to take into account patient-specific data for the determination of the exposure parameters of the X-ray procedure.

23. The X-ray system as claimed in claim 17, characterized in that the input arrangement (5) is or can be connected to a database or a measuring arrangement via an interface (15) so as to take into account radiologically relevant risk factors from the database for the determination of the exposure parameters of the X-ray procedure or, using the data processing system (11), it being possible to calculate radiologically relevant risk factors for the determination of the exposure parameters of the X-ray procedure on the basis of data determined by the measuring arrangement.

24. The X-ray system as claimed in claim 23 for generating X-ray images of a person to be X-rayed, characterized in that the database is an osteoporosis-statistics database for determining osteoporosis risk factors, the input arrangement (5) being designed such that the osteoporosis risk factors can be selected from the osteoporosis-statistics database on the basis of the age and sex of the person.

25. The X-ray system as claimed in claim 17, characterized in that the input arrangement (5) has a selection dialog box (17) for pathology-specific parameters, it being possible to select at least one pathology-specific parameter by means of input means (7) so as to take this parameter into account for the determination of the exposure parameters of the X-ray procedure.

26. A method for generating preferably digital X-ray images for adjusting exposure parameters of an X-ray procedure, in particular by using an apparatus as claimed in claim 17, it being possible to image the object to be X-rayed as a virtual model (12) in at least one position on a screen (10) of a visual display device (6), characterized in that the respective position of the object to be X-rayed is adjusted according to the model (12) of the screen in order to be displayed on the display device (6) by means of input means (7).

27. The method as claimed in claim 26, characterized in that a number of X-ray images (54, 55, 56) are made in sequence in different positions.

28. The method as claimed in claim 26, characterized in that a positional change of the model is animated.

29. The method as claimed in claim 28, characterized in that, in order to record X-ray images of a standing or a lying person, the model is turned about its longitudinal axis on the screen (10) during the transition from one position to a next position.

30. The method as claimed in claim 26, characterized in that a pathology-specific parameter, which is taken into account when specifying the exposure parameters of an X-ray procedure, is selected by means of input means (7) on the basis of a selection of pathology-specific parameters.

31. A computer program product for operating an X-ray system, which computer program product can be loaded into a data processing system, in particular into primary storage of the data processing system, and has at least one program code section which carries out a method as claimed in claim 26 when executed.

32. The computer program product as claimed in claim 31, characterized in that, taking account of a selected position of the model on the screen (10) and possibly on the basis of a selection of a selection region of the model assigned to a body part or an organ, the exposure parameters of an X-ray procedure, in particular to control the radiation dose, are calculated when the computer program product is executed on the data processing system.

33. A computer program product for operating an X-ray system, which computer program product can be loaded into a data processing system, in particular into primary storage of the data processing system, and has at least one program code section which carries out a method as claimed in claim 27 when executed.

34. A computer program product for operating an X-ray system, which computer program product can be loaded into a data processing system, in particular into primary storage of the data processing system, and has at least one program code section which carries out a method as claimed in claim 28 when executed.

35. A computer program product for operating an X-ray system, which computer program product can be loaded into a data processing system, in particular into primary storage of the data processing system, and has at least one program code section which carries out a method as claimed in claim 29 when executed.

36. A computer program product for operating an X-ray system, which computer program product can be loaded into a data processing system, in particular into primary storage of the data processing system, and has at least one program code section which carries out a method as claimed in claim 30 when executed.
